(19) United States
(12) Patent Application Publication
(10) Pub. No.: US 2006/0185448 A1
(43) Pub. Date: Aug. 24, 2006

(54) Biological specimen container cap with actuated access

(75) Inventors: Robert Sakal, Bolton, MA (US); Nathan Wood, Winchedon, MA (US)

Correspondence Address: Bingham McCuthen, LLP, Suite 1800, Three Embarcadero, San Francisco, CA, 94111-4067, US

(73) Assignee: Cytyc Corporation, Marlborough, MA (US)

(21) Appl. No.: 11/065,375

(22) Filed: Feb. 23, 2005

(51) Int. Cl. G01N 1/00; G01N 33/00; B01L 11/00

(52) U.S. Cl. 73/863; 422/63; 422/101

(57) ABSTRACT

Apparatus and method are provided for accessing a biological specimen container having a cap without opening the cap. A door covers an opening in the cap of the biological specimen container. The door may be biased to close the opening and to form a substantially fluid tight connection with a seal on the cap. An actuator is activated to open the door and an interior of the biological specimen container is accessible through the opening without contaminating an exterior of the biological specimen container or the cap.

### FIELD OF INVENTION

The present invention generally relates to devices and methods for storing and handling biological specimens, and more particularly, to devices and methods for accessing a biological specimen container having a cap

### DESCRIPTION OF RELATED ART

Various cup-like containers have been used to store biological specimens, especially fluid specimens. Because biological specimens can contain infectious agents and because cross contamination of biological specimens can lead to erroneous test results, removable caps have been used to close biological specimen cups. These caps have various fluid tight closing mechanisms, including snap fit and threaded mechanisms.

When collecting specimens, a user removes the cap from the container and deposits a collected biological specimen into the container. Then the cap is replaced, closing the container and preventing contamination of and by the sample. When the specimen is prepared for examination, a user removes the cap from the container and removes all or a portion of the specimen. Then the cap is once again replaced. Removing the cap of a container holding a biological specimen increases the risk of contamination of the specimen by the user or contamination of the user by the specimen.

Biological specimens are often prepared for examination by automated or semi-automated biological specimen collection and transfer systems. For instance, a slide can be prepared from a liquid cell suspension using an automated filter transfer technique, as disclosed in U.S. Pat. Nos. 6,572,824, 6,318,190, 5,772,818, 5,364,597, and 5,143,627, which are expressly incorporated herein by reference. Automated filter transfer devices are designed to cap and uncap biological specimen containers. This design requires a complex capping and uncapping mechanism, which can lead to increased system cost and potential reliability issues. This design also increases the risk that the contents of the container will be contaminated by the automated filter transfer devices.

### SUMMARY OF THE INVENTION

In one embodiment, a biological specimen collection and transfer system comprises a biological specimen slide processor, a biological specimen container configured for use with the slide processor, a cap configured for use with the biological specimen container, where the cap defines an opening for accessing an interior of the biological specimen container, and a door for closing the opening.

In accordance with another embodiment, a cap configured for use with a biological specimen container comprises a cap body having a bottom surface and a peripheral wall extending from the bottom surface, where the bottom surface defines an opening, and a door coupled to the bottom surface, where the door seals the biological specimen container when closed and allows access to an interior of the biological specimen container when opened.

In various embodiments the door is biased to close the opening, and the system may further comprise a seal, which forms a fluid tight connection between the door and the cap. In various embodiments, the system may further comprise an actuator, which is activated to open the door. In various embodiments, a biological specimen is contained in the biological specimen container, and an interior of the biological specimen container is accessible through the opening without contaminating an exterior of the biological specimen container and the cap.

In another embodiment, a method of accessing a fluid in a biological specimen container without removing a cap comprises opening a door of the cap, accessing the fluid inside the biological specimen container through the opened door, and closing the door. The method may further comprise accessing the fluid through the opened door without contaminating an exterior of the biological specimen container and the cap.

### BRIEF DESCRIPTION OF THE DRAWINGS

In order to better understand and appreciate the invention, reference should be made to the accompanying drawings that illustrate exemplary embodiments thereof, in which similar elements are referred to by common reference numerals, and in which:

FIG. 1 is a perspective view of a biological specimen container and a cap for accessing the container without opening the cap;

FIG. 2 is a front view of the biological specimen container and cap of FIG. 1;

FIG. 3 is a perspective view of the biological specimen container of FIG. 1;

FIG. 4 is a perspective view, from the bottom, of the cap of FIG. 1;

FIG. 5 is a cross sectional view through the line x-x in FIG. 2;

FIG. 6 is a detailed perspective view of a hinge, a vertical gear, and an axle of the cap of FIG. 1;

FIG. 7 is a detailed perspective view of vertical and horizontal gears of the cap of FIG. 1, showing the relative position of a Phillips screwdriver tip;

FIG. 8 is a schematic view of an automatic biological specimen collection and transfer system, which is configured to utilize the biological specimen container of FIG. 1;

FIG. 9 is a detailed perspective view of a top surface of the cap of FIG. 1 showing the interaction of a locking hook and a wedge-shaped spring loaded latch;

FIG. 10 is a perspective view of a biological specimen container and a cap, according to another embodiment, for accessing the container without opening the cap;

FIG. 11 is a detailed perspective view of a hinge and an axle of the cap of FIG. 10, which is shown in shadow;

FIG. 12 is a perspective view of a biological specimen container and a cap, according to another embodiment, for accessing the container without opening the cap;

FIG. 13 is a bottom view of the cap of FIG. 12 with an opening, an O-ring, and a hub shown in shadow; and

FIG. 14 is a detailed perspective view, from the bottom, of the cap of FIG. 12, showing the interaction of a partially wedge-shaped spring loaded bolt, an aperture, and a stop to lock the door in a closed position.

### DETAILED DESCRIPTION OF ILLUSTRATED EMBODIMENTS

In the following description of the illustrated embodiments, it will be understood by those skilled in the art that the drawings and specific components thereof are not necessarily to scale, and that various structural changes may be made without departing from the scope or nature of the embodiments.

A biological specimen container and cap are disclosed for accessing a biological specimen container having a cap without opening the cap. The cap defines an opening through which the container is accessed. The cap includes a door, which is biased to close the opening, and a seal, which forms a fluid tight connection between the cap and the door. The cap also includes an actuator, which opens the door to allow access to the container without opening the cap. Additional details regarding the container and cap components and their operation follows.

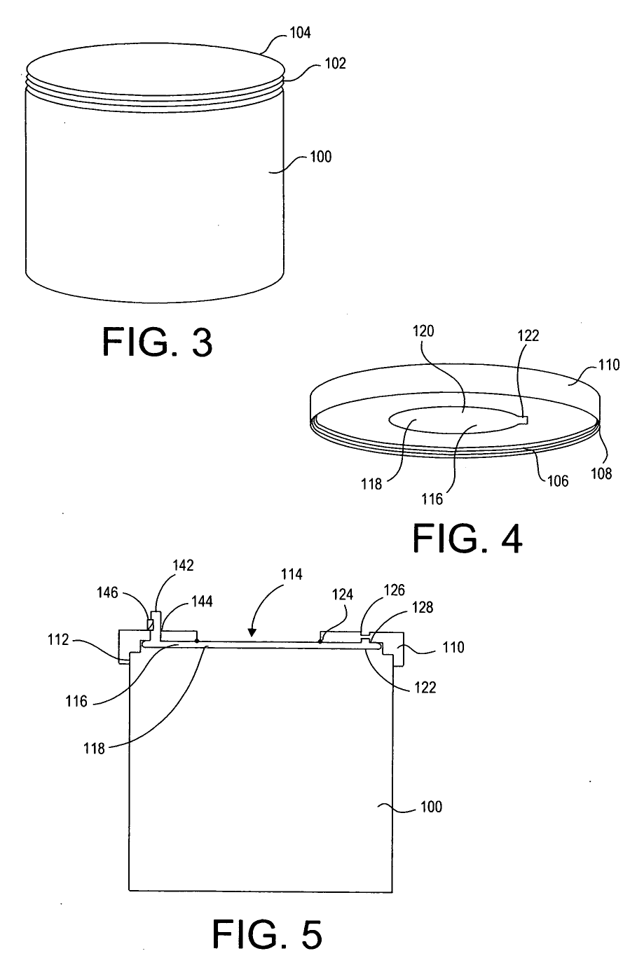

Referring to FIGS. 3 and 4, a biological specimen container 100 according to one embodiment includes a first threaded ring 102 disposed at its open top end 104 and configured to match a second threaded ring 106 disposed on the bottom end 108 of a circular cap 110. The first and second threaded rings 102 and 106 form a fluid tight seal 112 between the container 100 and the cap 110, as shown in FIG. 5. Both the container 100 and the cap 110 are molded from plastic.

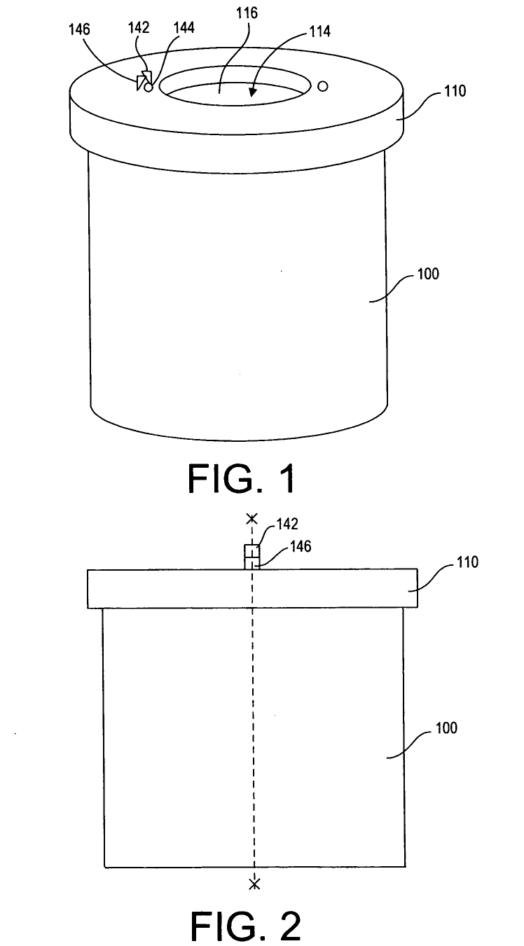

Referring to FIGS. 1, 2 and 5, the cap 110 defines a circular opening 114 in the middle of and concentric with the cap 110. The opening 114, which is formed in the cap 110 during molding, allows the container 100 to be accessed from above without opening the cap 110. The cap 110 also includes a door 116, which is biased to close the opening 114.

The door 116 includes a planar member 118 made of rigid plastic in the shape of a circle 120 with a projection 122 extending from one side of the circle 120. The circle 120 is slightly larger than the opening 114 and therefore, able to close the opening 114. The door 116 sits in a ring-shaped recess 120, which is formed in the bottom of the cap 110, such that when the door 116 is in the closed position, the bottom of the door 116 is approximately flush with the bottom of the cap 110. When the door 116 is in the closed position, the top of the door 116 abuts an O-ring 124 disposed on the bottom surface of the cap 110 around the opening 114, forming a fluid tight connection between the door 116 and the cap 110.

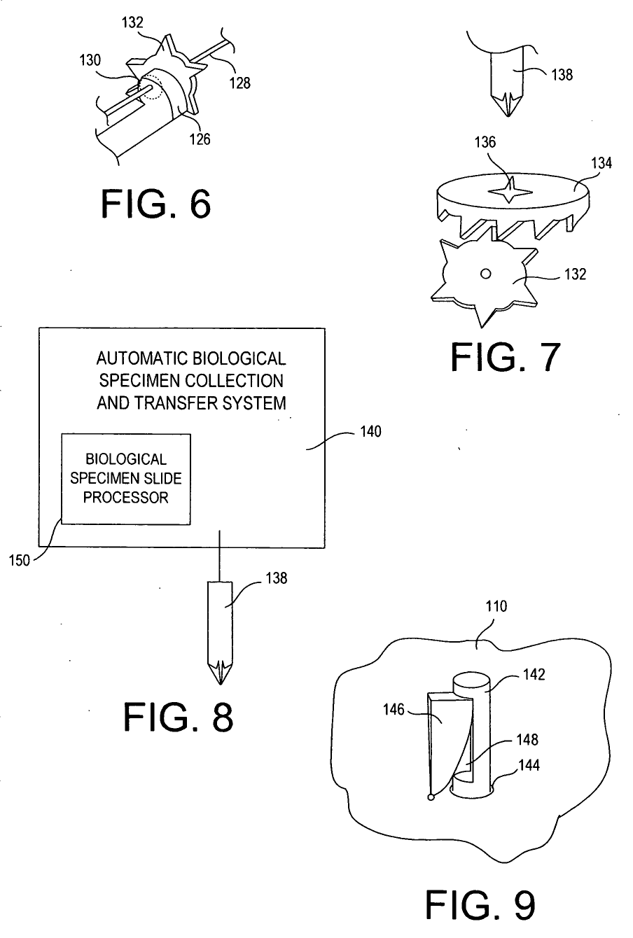

The door 116 has a hinge 126 formed on the end of the projection 122. The hinge 126 is attached to an axle 128, which is attached to the cap 110, as shown in FIG. 6. The hinge is attached to a coil spring 130, which is also attached to the axle 128 and biases the door 116 in the closed position. The hinge is also attached to a vertical gear 132, which is juxtaposed to a horizontal gear 134, as shown in FIG. 7. This arrangement of gears translates rotation of the horizontal gear 134 to a rotation of the hinge 126, which opens the door 116. The top surface of the horizontal gear 134 defines a cruciform depression 136 in its center, which facilitates rotation of the horizontal gear 134 by a Phillips screwdriver tip 138 attached to an automatic biological specimen collection and transfer system 140, as shown in FIG. 8.

Referring to FIGS. 1, 5, and 9, the door 116 also includes a locking hook 142 disposed on the portion of the top surface of the circle 120 that overlaps the cap 110. When the door is closed, the locking hook 142 passes through a hole 144 defined by the cap 110 and protrudes out of the top of the cap 110. As the locking hook 142 passes through the top of the cap 110, it compresses a wedge-shaped spring loaded latch 146 disposed near the hole 144 until the latch 146 is positioned next to a notch 148 defined by the hook 142. The latch 148 then rebounds into the notch 148, locking the hook 142 and the door 116 to which it is connected in the closed position.

In operation, the biological specimen collection and transfer system 140 positions the Phillips screwdriver tip 138 near the cruciform depression 136 from above the cap 110. The system 140 then depresses the spring loaded latch 146 and rotates the screwdriver tip 138 in a clockwise direction, opening the door 116 via the gears 132 and 134 and tensioning the coil spring 130. A biological specimen slide processor 150, which is part of the system 140, accesses the biological specimen container 100. Finally, the system 140 rotates the screwdriver tip 138 in a counterclockwise direction, and the tension from the coil spring 130 closes the door 116. In such a way, the system 140 accesses the container 100 without removing the cap 110.

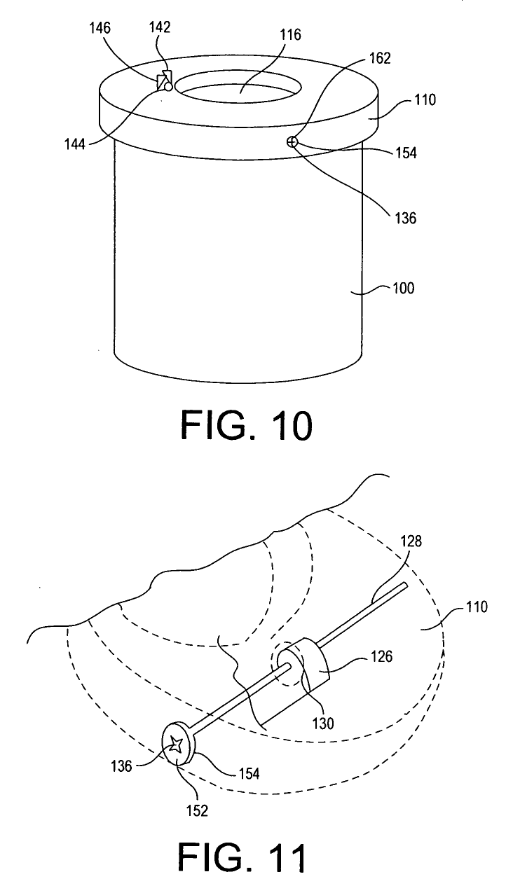

Persons skilled in the art will appreciate that other mechanisms can be used to open the door 116. For example, FIGS. 10 and 11 show another embodiment of a cap 110 with a door 116. This door 116 is attached to an axle 128. The axle 128 is attached to a coil spring 130, which is also attached to the cap 110. One end of the axle 128 is attached to a button 152, which is fitted in a slot 154 in the cap 110 such that the button 152 is accessible from outside of the cap 110 and flush with the side surface of the cap 110. The outside facing surface of the button 152 defines a cruciform depression 136, which facilitates rotation of the axle 128 and the attached hinge 126 and coil spring 130 by a Phillips screwdriver tip 138 attached to an automatic biological specimen collection and transfer system 140. The cap 110 also includes the locking mechanism described above.

In operation, the biological specimen collection and transfer system 140 positions the Phillips screwdriver tip 138 near cruciform depression 136 from the side of the cap 110. The system 140 then depresses the spring loaded latch 146 and rotates the screwdriver tip 138 in the counterclockwise direction, rotating the axle 128, opening the door 116 and tensioning the coil spring 130. The biological specimen slide processor 142 accesses the biological specimen container 100. Finally, the system 140 rotates the screwdriver tip 138 in a clockwise direction, and the tension from the coil spring 130 closes the door 116. In such a way, the system 140 accesses the container 100 without removing the cap 110.

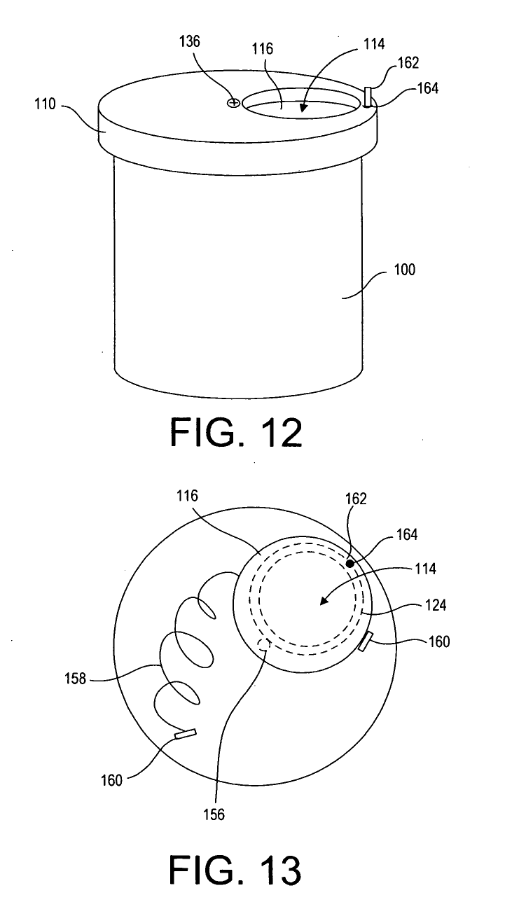

Referring to FIGS. 12 and 13, another embodiment of a cap 110 defines an opening 114, which is offset from the center of the cap 110. The cap 110 also includes a door 116, which includes a planar member 118 made of rigid plastic in the shape of a circle 120. The circle 120 is slightly larger than the opening 114, so that when the door 116 is positioned over the opening 114, the opening 114 is closed. The planar member 118 is attached to a hub 156, which pierces the center of the cap 110 and rotates. The planar member 118 is also attached to a spring 158, which is attached to the bottom of the cap 110 and biases the door 116 in the closed position. The hub 156 defines a cruciform depression 136 on its top surface to facilitate rotation of the hub 156 with a Phillips screwdriver tip 138.

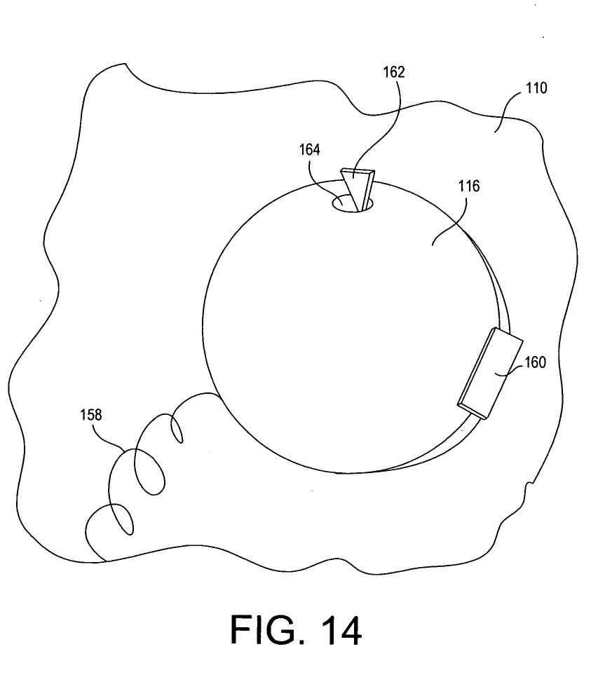

The cap 110 includes two stops 160 disposed on its bottom surface, which limit the rotation of the hub 156 and consequently, the movement of the door 116. The cap 110 also includes a partially wedge-shaped spring loaded bolt 162, which pierces the cap 110 at a point opposite of the opening 114 form the hub 156. The spring loaded bolt 162 is biased in a position in which it protrudes from the bottom of the cap 110. The door 116 also includes an aperture 164 that overlaps the spring loaded bolt 120 when the door is closed. In that position, the spring loaded bolt 162 passes through the aperture 164, locking the door 116 in the closed position, as shown in FIG. 14.

In operation, the biological specimen collection and transfer system 140 positions the Phillips screwdriver tip 138 near the cruciform depression 136 from above the cap 110. The system 140 then pulls the spring loaded bolt 162 up and out of the aperture 164 and rotates the screwdriver tip 138 in a clockwise direction until it hits a stop 160, thereby opening the door 116 and compressing the spring 158. Then a biological specimen slide processor 150, which is part of the system 140, accesses the biological specimen container 100. Finally, the system 140 rotates the screwdriver tip 138 in a counterclockwise direction, and the tension from the spring 158 closes the door 116 until it hit the other stop 160. In such a way, the system 140 accesses the container 100 without removing the cap 110.

What is claimed:

1. A biological specimen collection and transfer system, comprising:
a biological specimen slide processor;
a biological specimen container configured for use with the slide processor;
a cap configured for use with the biological specimen container and defining an opening for accessing an interior of the biological specimen container; and
a door operatively coupled to the cap for closing the opening.

2. The system of claim 1, wherein the door is biased to close the opening upon activation.

3. The system of claim 1, further comprising a seal that forms a substantially fluid tight connection between the door and the cap.

4. The system of claim 1, further comprising an actuator for opening the door.

5. The system of claim 4, wherein the actuator is biased to open the door upon activation.

6. The system of claim 1, fwherein the biological specimen container interior is accessible through the opening without contaminating an exterior of the biological specimen container or the cap.

7. A cap configured for use with a biological specimen container, the cap comprising:
a cap body having a bottom surface and a peripheral wall extending from the bottom surface;
the bottom surface defining an opening; and
a door operatively coupled to the bottom surface,
the door substantially sealing the biological specimen container when closed, and allowing access to an interior of the biological specimen container when opened.

8. The cap of claim 7, wherein the door is biased in a closed position.

9. The cap of claim 7, further comprising a seal forming a substantially fluid tight connection between the door and the cap.

10. The cap of claim 7, further comprising an actuator for opening the door.

11. The cap of claim 7, wherein an interior of the biological specimen container is accessible through the opening without contaminating an exterior of the biological specimen container or the cap.

12. A method of accessing a fluid in a biological specimen container without removing a cap, the method comprising:
opening a door of the cap;
accessing the fluid inside the biological specimen container through the opened door; and
closing the door.

13. The method of claim 12, further comprising accessing the fluid through the opened door without contaminating an exterior of the biological specimen container or the cap.
